(19) United States
(12) Patent Application Publication
(10) Pub. No.: US 2024/0325624 A1
(43) Pub. Date: Oct. 3, 2024

(54) TELESCOPIC TUBING EXTENSION SET

(71) Applicant: CareFusion 303, Inc., San Diego, CA (US)

(72) Inventors: Sumit BALI, Midnapur (IN); Apurva Vikram AGARWAL, Lucknow (IN)

(21) Appl. No.: 18/602,650

(22) Filed: Mar. 12, 2024

Related U.S. Application Data
(60) Provisional application No. 63/492,897, filed on Mar. 29, 2023.

(51) Int. Cl. A61M 5/14

(52) U.S. Cl. CPC A61M 5/1413

(57) ABSTRACT

Extendable tubing extension sets are disclosed having a sleeve and a tubing that can move telescopically in a longitudinal direction relative to each other while maintaining the patency of a fluid pathway through the telescopic tubing extension set when the tubing and the sleeve are moved in a direction toward each other to a first configuration, and in a direction away from each other toward a second configuration, and where the tubing is positioned within a tubing lumen of the sleeve and a fluid pathway can extend through any of a fluid passage of the tubing and the tubing lumen of the sleeve.

### CROSS REFERENCE TO RELATED APPLICATION

This application claims the benefit of priority under 35 U.S.C. § 119 to Provisional application for Patent Ser. No. 63/492,897, entitled “TELESCOPIC TUBING EXTENSION SET,” filed on Mar. 29, 2023, the entire contents of which is incorporated herein by reference.

### BACKGROUND

The present disclosure relates generally to medical fluid connectors and, more particularly, to telescopic tubing extension sets with a sleeve and tubing that are movable relative to each other.

Medical connections are widely used in fluid delivery systems such as those used in connection with intravenous (IV) fluid lines, blood access, hemodialysis, peritoneal dialysis, enteral feeding, drug vial access, and other procedures.

In some instances, the medical connection can become dislodged or disconnected in an unintended manner. For example, medical tubing of an IV set that is coupled to a catheter can become dislodged when an unintended or unexpected forces is exerted upon a portion of the IV set such as the catheter or the tubing, which may exceed the design limitations of the catheter securement method or another portion of the IV set. An unintended or unexpected force can be applied to the tubing and/or catheter when the patient moves or rolls over within a bed, or when the tubing or another portion of an intravenous set become caught on a portion of the bed, such as the railing, or when a patient is panicking, disoriented, or fidgeting to such an extent that the medical tubing is unintentionally or intentionally pulled away from the patient or away from the medical equipment coupled to the tubing.

### SUMMARY

In accordance with at least some embodiments disclosed herein is the realization that unintended dislodgement or disconnection of a medical connection, such as a medical tubing, can result in injury to a patient or a caregiver, such as by depriving the patient of a medicament, increasing the potential for infection to the patient, and exposing the caregiver to harmful medicaments.

Accordingly, aspects of the present disclosure provide telescopic tubing extension sets comprising a sleeve having a first end, a second end, and an inner surface forming a tubing lumen extending through the first and second ends of the sleeve, the second end of the sleeve comprising a locking feature configured to couple with a tubing coupler, and the first end of the sleeve comprising a seal, and a tubing having a proximal end, a distal end, an inner surface forming a fluid passage extending through the proximal and distal ends, a collar extending radially outward from an outer surface of the tubing, and a distal end portion formed by the outer surface of the tubing defining a cross-sectional width of the tubing that decreases in a direction from the collar toward the distal end of the tubing, wherein a distal end portion of the tubing is positioned within the tubing lumen of the sleeve such that the seal is engaged against the tubing and the tubing is longitudinally movable relative to the sleeve.

The present disclosure can also provide a telescopic tubing extension set systems comprising a tubing coupler having an outer surface and an inner surface, wherein the inner surface forms a fluid channel extending through the tubing coupler, and a telescopic tubing extension set comprising a sleeve, a tubing, and a seal, the sleeve comprising a first end engaged against the seal, a second end having a locking feature configured for coupling with the tubing coupler, and a tubing lumen extending through the first and second ends of the sleeve, and the tubing positioned within the tubing lumen and having a distal end configured for removably coupling with the fluid channel of the tubing coupler, an inner surface forming a fluid passage, and an outer surface engaged against the seal, wherein, in a first orientation of the telescopic tubing extension set, the locking feature is engaged against the tubing coupler to resist separation of the sleeve from the tubing coupler, and the distal end of the tubing is positioned within the fluid channel of the tubing coupler to form a fluid pathway between the fluid channel of the tubing coupler and the fluid passage of the tubing, and in a second orientation of the telescopic tubing extension set, the tubing is longitudinally moved in a direction away from the tubing coupler such that the distal end of the tubing is withdrawn from the tubing coupler and positioned between the first and second ends of the sleeve to form a fluid pathway between the fluid channel of the tubing coupler, the tubing lumen of the sleeve, and the fluid passage of the tubing.

In some instances, the present disclosure provides methods for providing a telescopic tubing extension set, the method comprising providing a sleeve with an inner surface forming a tubing lumen between a first end and a second end of the sleeve, positioning a tubing within the tubing lumen of the sleeve such that tubing is longitudinally movable within the tubing lumen in a first direction, such that a distal end of the tubing moves toward the second end of the sleeve, and a second direction, such that a distal end of the tubing moves away the second end of the sleeve, and positioning a seal between the sleeve and the tubing to form resist movement of a fluid between an outer surface of the tubing and the inner surface of the sleeve.

Accordingly, the present application addresses several operational challenges encountered in prior fluid connections and provides numerous improvements that enable the user to more easily provide a fluid pathway, and to increase safety and efficacy in maintaining a fluid pathway and fluid delivery. In some aspects, the present disclosure provides a telescopic tubing extension set that can resist separation or damage to a fluid pathway by permitting a portion of the structure forming the fluid pathway to extend in length.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates a telescopic tubing extension set in use with an IV set coupled to a patient, in accordance with aspects of the present disclosure.

FIG. 2 illustrates a perspective view of a telescopic tubing extension set in a first orientation coupled with a catheter, in accordance with aspects of the present disclosure.

FIG. 3 illustrates a perspective view of the telescopic tubing extension set of FIG. 2 in a second orientation, in accordance with aspects of the present disclosure.

FIG. 4 illustrates a perspective exploded view of the telescopic tubing extension set, in accordance with aspects of the present disclosure.

FIG. 5 illustrates a cross-sectional view of the telescopic tubing extension set of FIG. 4, in accordance with aspects of the present disclosure.

FIG. 6 illustrates a detail cross-sectional view of the telescopic tubing extension set, in accordance with aspects of the present disclosure.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

In accordance with some embodiments, the present application discloses various features and advantages of a telescopic tubing extension set. The telescopic tubing extension set can provide for efficient and safe maintenance of a fluid pathway, such as a fluid pathway used for transferring medical fluids toward or away from a patient. The telescopic tubing extension set can maintain the structural integrity and sterility of a fluid pathway by resisting unintended disconnection when a pulling or tension force is applied to the telescopic tubing extension set or another device that forms the fluid pathway, such as when a patient moves or when the medical tubing is pulled away from the patient.

The telescopic tubing extension set can also prevent injury to a patient or a caregiver by preventing unintended disconnection of the fluid pathway from the patient, which could deprive the patient of a medicament and could expose the patient or another person to the medicament. The telescopic tubing extension set can also reduce labor and cost associated with fluid transfer by avoiding unintended disconnection of the fluid pathway.

The telescopic tubing extension set includes a sleeve and tubing that can move longitudinally relative to each other, thereby providing a telescopic function in which the tubing can move through a portion of the length of the sleeve to increase a length of the fluid pathway through telescopic tubing extension set.

At least a portion of the length of the tubing is positioned within the sleeve such that the tubing is concentrically located within the sleeve when a cross-sectional profile of the telescopic tubing extension set is viewed in a plane that is transverse to a longitudinal axis of the telescopic tubing extension set.

The tubing positioned within the sleeve forms a fluid pathway when the telescopic tubing extension set is in a first orientation, a second orientation, and when the telescopic tubing extension set is moving between the first and second orientations.

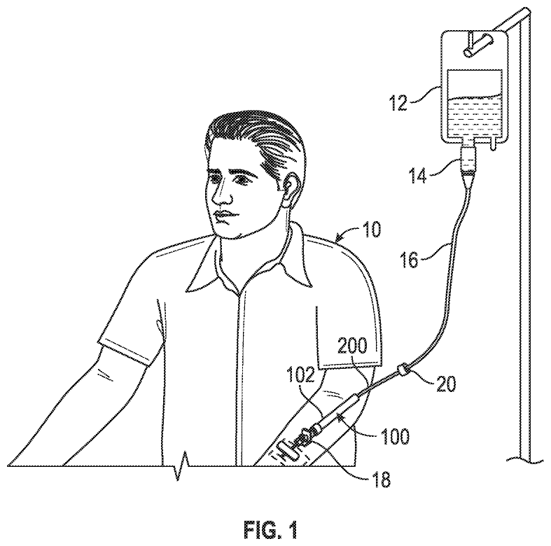

An example of the telescopic tubing extension set in the first orientation, in accordance with aspects of the present disclosure, is illustrated in FIG. 1. The telescopic tubing extension set is coupled with tubing of an IV set, which is being used to direct a fluid to a patient 10. The IV set can include a medicament bag 12, a drip chamber 14, tubing 16, and an IV catheter 18.

The telescopic tubing extension set 100 fluidly connects the tubing 16 to the IV catheter 18. Although the telescopic tubing extension set 100 is illustrated being coupled along a fluid pathway of an IV set, between a medicament bag 12 and a patient 10, it should be understood that the telescopic tubing extension set 100 can be connected within other fluid pathways, such as between a patient and an IV pump or between a patient and a dialysis machine. The telescopic tubing extension set 100 can also be connected along another portion of a fluid pathway. For example, the telescopic tubing extension set 100 can be connected along a proximal portion of the fluid pathway, such as being connected between the tubing 16 and the medicament bag 12 or other fluid therapy device. The telescopic tubing extension set 100 can also be connected along other fluid pathways, such as along a portion of a feeding tube pathway.

The telescopic tubing extension set 100 can form all or a portion of a fluid pathway. In some aspects, tubing of the telescopic tubing extension set 100 can extend from a fluid source to the sleeve; however, the present disclosure also contemplates that the tubing of the telescopic tubing extension set can be coupled to another length of tubing or another fluid device using a connector. An example of a connector 20 for coupling tubing of the telescopic tubing extension set to tubing of the IV set is illustrated in FIG. 1.

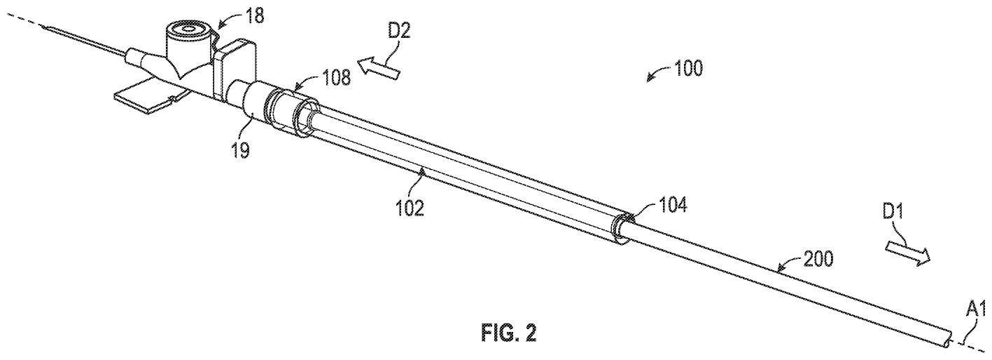

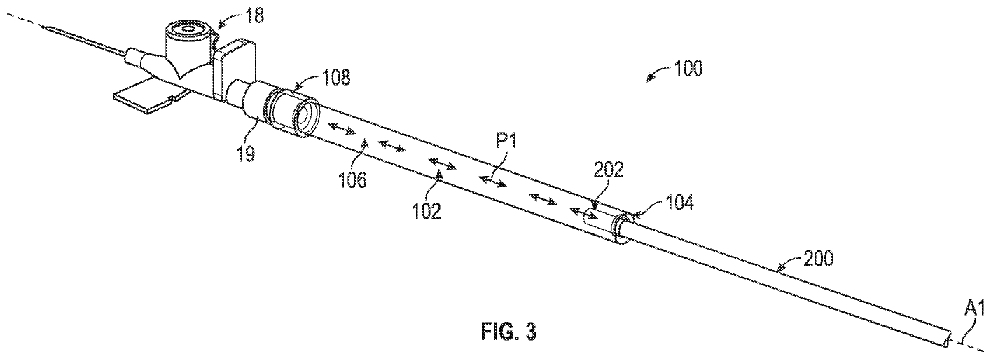

The telescopic tubing extension set 100 can move between a first orientation and a second orientation. FIG. 2 illustrates the telescopic tubing extension set 100 in the first orientation and FIG. 3 illustrates the telescopic tubing extension set 100 in the second orientation. Although the telescopic tubing extension set 100 is illustrated as being coupled to an IV catheter 18, it should be understood that the telescopic tubing extension set 100 can be coupled to another portion of an IV set, another fluid transfer device, or a tubing coupler 19 that can be coupled between the telescopic tubing extension set 100 and another device, such as the IV catheter 18.

The telescopic tubing extension set 100 includes a sleeve 102 and tubing 200 positioned within the sleeve 102. The sleeve 102 and tubing 200 are longitudinally movable relative to each other to permit the telescopic tubing extension set 100 to move between the first and second orientations.

The sleeve 102 is illustrated as being comprised of a material that is transparent to illustrate the position of the tubing 200 relative to the sleeve 102, however, the present disclosure contemplates that any of the sleeve 102 and/or the tubing 200 can comprise a material that is transparent or opaque.

In the first orientation, the tubing 200 extends through an opening at a first end 104 of the sleeve, and along a tubing lumen 106 formed by an inner surface of the sleeve. A distal end 202 of the tubing is coupled to the tubing coupler 19, and a second end 108 of the sleeve is also coupled to the tubing coupler 19.

In the first orientation of the telescopic tubing extension set 100, the fluid pathway extends through the tubing coupler 19 and a fluid passage formed by the inner surface of the tubing 200.

The distal end 202 of the tubing and the second end 108 of the sleeve are coupled to the tubing coupler 19. The second end 108 of the sleeve includes a locking feature configured to couple with the tubing coupler 19 and resist separation of the sleeve 102 therefrom.

The distal end 202 of the tubing is also coupled to the tubing coupler 19 and is configured to permit separation or withdrawal of the distal end 202 the tubing therefrom when a force exceeds a threshold.

When a force on the telescopic tubing extension set 100 exceeds the threshold, the telescopic tubing extension set 100 can move from the first orientation toward the second orientation. The force can be applied to any of the sleeve 102 and/or the tubing 200. The force can be in any direction that causes the sleeve 102 and the tubing 200 to be pulled away from each other. For example, a force that causes the telescopic tubing extension set 100 to move from the first orientation to the second orientation can be caused by the tubing 200 being pulled in a direction D1 away from the first end 104 of the sleeve and can be caused by the sleeve 102 being pulled in a direction D2 away from the tubing 200.

In some embodiments of the present disclosure, the threshold force that can cause the tubing 200 to separate from or withdraw from the tubing coupler 19 is between approximately one pound of force and approximately ten pounds of force, or the threshold force can be between approximately three pounds of force and approximately 7 pounds of force. In some embodiments, the telescopic tubing extension set 100 is configures so that the tubing 200 separates from or withdraws from the tubing coupler 19 when the force exceeds approximately five pounds of force.

When a force on the telescopic tubing extension set 100 exceeds the threshold, the telescopic tubing extension set 100 moves toward the second orientation, illustrated in FIG. 3, wherein the tubing 200 is longitudinally moved within the sleeve 102 in a direction away from the second end 108 of the sleeve.

The tubing 200 can move along a longitudinal axis A1 defined between the first and second ends 104, 108 of the sleeve. In the second orientation, the tubing 200 can move away from the second end 108 of the sleeve by a distance that is less than a length of the sleeve 102, such that the distal end 202 of the tubing remains positioned within the tubing lumen 106 of the sleeve.

In some embodiments of the present disclosure, the length of the sleeve 102 is defined between the first and second ends 104, 108 of the sleeve and is between approximately one inch and approximately eight inches, or is between approximately two inches and approximately is inches. In some embodiments, the length of the sleeve 102 is approximately 4 inches.

In the second orientation of the telescopic tubing extension set 100, the fluid pathway P1 extends through the tubing coupler 19, the tubing lumen 106 of the sleeve, and the fluid passage formed by the inner surface of the tubing 200.

In the first and second orientation of the telescopic tubing extension set 100, the sleeve 102 is coupled to the tubing coupler 19 by a locking a locking feature 110 at the second end 108 of the sleeve. The locking feature 110 is configured to permit coupling and decoupling of the second end 108 of the sleeve to the tubing coupler 19, when coupling or decoupling is intended. However, the locking feature 110 also resists unintended separation or decoupling of the sleeve 102 from the tubing coupler 19, such as when a force is exerted on the telescopic tubing extension set 100 or when the telescopic tubing extension set 100 is moved from the first orientation toward the second orientation.

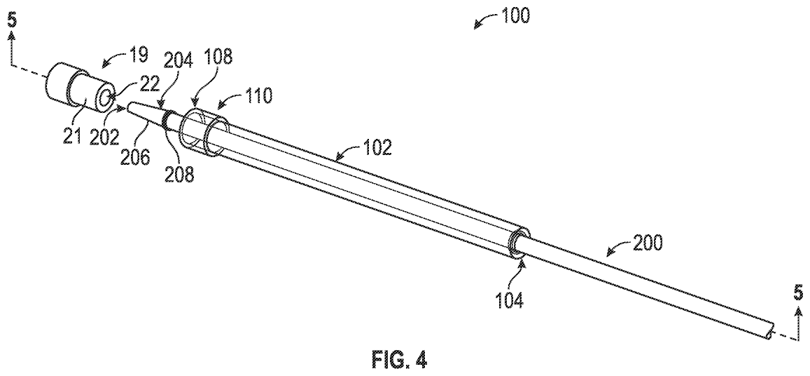

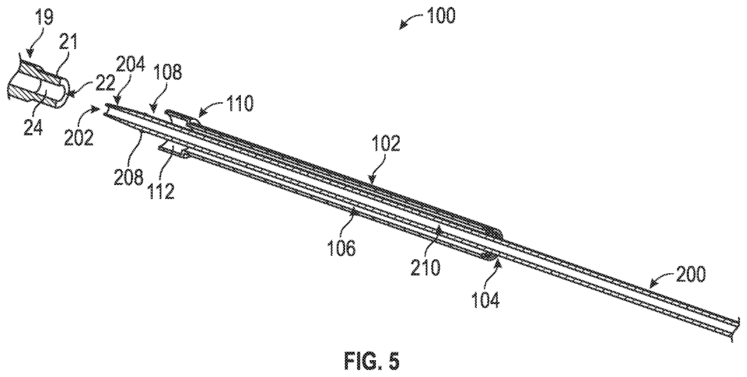

Referring to FIGS. 4 and 5, the locking feature 110 can be formed at the second end 108 of the sleeve by any structure or feature configured to resist unintended separation of the sleeve 102 from the tubing coupler 19. In some aspects of the present disclosure, the locking feature is configured to resist separation of the sleeve 102 from the tubing coupler 19 before separation of the tubing 200 from the tubing coupler 19. In some embodiments of the present disclosure, the locking feature is configured to resist separation of the sleeve 102 from the tubing coupler 19 until a separation force exerted on the sleeve is greater than a threshold force for moving the telescopic tubing extension set 100 from the first orientation toward the second orientation.

The threshold separation force for the connection between the sleeve 102 and the tubing coupler 19 is configured for forces in a longitudinal direction. As such, a rotational force applied to the sleeve 102 or tubing coupler 19 can permit unscrewing or separation of the sleeve 102 from the tubing coupler 19, where the rotational force is less than the threshold separation force for the connection between the sleeve 102 and the tubing coupler 19.

The locking feature 110 can be formed as a thread that extends radially inward from the inner surface 112 of the sleeve and is configured to couple with a mating thread of the tubing coupler 19. The locking feature 110 can also be configured as any of a tab extending from the sleeve 102 or a groove extending into the sleeve 102 and configured to engage against the other or the tab or groove of the tubing coupler 19. In instances where the locking feature is a thread or tab and groove, coupling of the sleeve 102 to the tubing coupler 19 can be achieved by less than 180 degrees of rotation between the sleeve 102 and the tubing coupler 19.

The present disclosure also contemplates embodiments in which the locking feature is another structure or function, such as an interference fit or snap connection between the sleeve 102 and the tubing coupler 19. In some embodiments of the present disclosure, the locking feature is formed by the inner surface 112 at the second end of the sleeve having a cross-sectional width that is approximately equal to a cross-sectional width formed by an outer surface 21 of the tubing coupler 19.

When the sleeve 102 is coupled to the tubing coupler 19, the inner surface of the sleeve is engaged against an outer surface 21 of the tubing coupler. In the first orientation of the telescopic tubing extension set 100, the locking feature 110 of the sleeve is coupled to the tubing coupler 19, and the distal end 202 of the tubing is positioned within a fluid channel 22 formed by an inner surface 24 of the tubing coupler.

To position the distal end 202 of the tubing within the fluid channel 22 of the tubing coupler, and form a fluid seal therebetween, a distal end portion 204 of the tubing can have tapered outer surface 206.

The tapered surface along the distal end portion of the tubing 200 can be formed by the outer surface of the tubing having a cross-sectional width that decreases to taper toward the distal end 202. The tapered distal end portion of the tubing can, in some embodiments of the telescopic tubing extension set 100, form a male luer taper configured to be received within and engage against a female luer taper formed by the inner surface 24 of the tubing coupler.

The distal end portion of the tubing 200 can also include a collar 208 configured resist or limit longitudinal movement of the tubing 200 relative to the sleeve 102 when the telescopic tubing extension set 100 moves toward the second orientation.

The collar 208 can resist or limit longitudinal movement of the tubing 200 by engaging against a portion of the sleeve 102 or another portion of the telescopic tubing extension set 100 when the tubing 200 moves proximally toward the first end 104 of the sleeve.

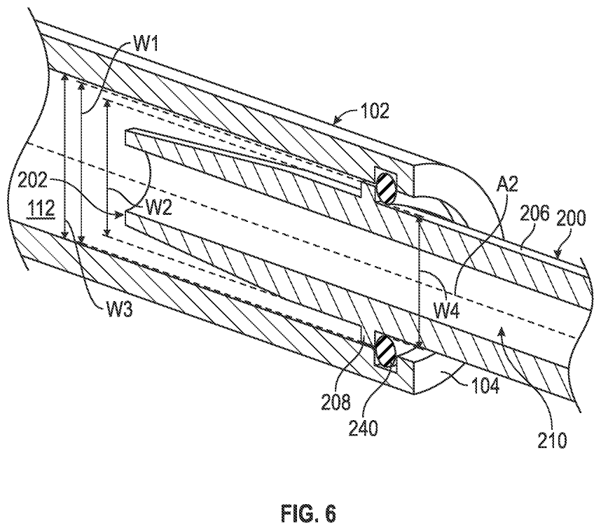

Referring to FIG. 6, the collar 208 is formed as a protrusion or wall that extends radially outward from the outer surface 206 of the tubing in a direction away from a longitudinal axis A2 of the fluid passage 210.

The collar 208 has a length that extends around the perimeter of the tubing 200. The length of the collar 208 is equal to or greater that the circumference of the tubing 200 such that the collar 208 extends around the entire perimeter of the tubing 200. In some embodiments, however, the collar 208 is extends around a portion of the perimeter of the tubing 200. In some embodiments of the present disclosure, the collar 208 is discontinuous around the perimeter of the tubing 200 and/or the collar 208 forms more than one discontinuous protrusion or wall extending from the outer surface 206 of the tubing.

To resist or limit longitudinal movement of the tubing 200 relative to the sleeve 102, an outer surface of the collar 208 defines a width W1 that is greater than a width W2 defined by the outer surface of the tubing 200 adjacent to the collar 208 and is less than a width W3 defined by the inner surface 112 of the sleeve. As the tubing 200 is longitudinally retracted in a proximal direction toward the first end 104 of the sleeve, the collar 208 can engage against a portion of the sleeve 102 or another portion of the telescopic tubing extension set 100.

Referring to FIG. 6, a cross-sectional view of a portion of the tubing 200 and the sleeve 102 are illustrated when the telescopic tubing extension set 100 is in the second orientation. The distal end portion 204 of the tubing is withdrawn from the fluid channel 22 of the fluid coupler, and the tubing 200 is longitudinally retracted toward the first end 104 of the sleeve.

Further retraction of the tubing 200 in the proximal direction is resisted when the collar 208 engages against a seal 240 positioned between the sleeve 102 and the tubing 200. The seal 240 is positioned at the first end 104 of the sleeve or adjacent to the first end 104 of the sleeve to permit the tubing 200 to move along a length of the tubing between the seal 240 and the second end 108 of the sleeve.

The seal 240 is also configured to resist movement of a fluid between the sleeve 102 and the tubing 200. The seal 240 can resist movement of fluid between the sleeve 102 and the tubing 200 when the telescopic tubing extension set 100 is in the second orientation, where a portion of the fluid pathway P1 extends through the tubing lumen 106 of the sleeve.

To resist movement of a fluid between the sleeve 102 and the tubing 200, the seal 140 has an inner surface forming an opening through the seal 140 and configured to engage against the tubing 200, and an outer surface configured to engage against the sleeve 102. In some embodiments of the present disclosure, the seal 240 can positioned at least partially within a groove or ledge formed by the sleeve 102, which can resist movement of the seal 140 when any of the tubing 200 or sleeve 102 are moved relative to each other.

To limit longitudinal movement of the tubing 200 toward the first end 104 of the sleeve, a portion of the sleeve extends into the tubing lumen 106 of the sleeve and is configured to be engaged against by the collar 208.

To resist movement of a fluid between the sleeve 102 and the tubing 200 and/or limit longitudinal movement of the tubing 200 toward the first end 104 of the sleeve, the opening of the seal 240 has a cross-sectional width W4 that is greater than the width W2 defined by the outer surface of the tubing 200 and less than the W1 defined by the collar 208.

The seal 240 can be an o-ring, another ring or toroid shaped seal, or another material configured to resist movement of a fluid. It is also contemplated by the present disclosure that in some embodiments, the sleeve 102 can have a groove or channel that extends along the inner surface 112 of the sleeve and is configured to receive a collar or protrusion of the tubing 200 therein.

In some embodiments, the sleeve can have a tubing stopping feature that is separate from the seal 240. In some aspects, the tubing stopping feature can be formed by a ledge or ridge that extends toward the tubing 200 and is configured to be engaged against by the collar 208, and the seal 240 can be positioned between the tubing stopping feature and the first end 104 of the sleeve. It should also be understood that the present disclosure contemplates other embodiments providing a tubing stopping feature and a seal between the sleeve 102 and the tubing 200. In some examples of the present disclosure, a seal can be positioned between the sleeve 102 and the tubing 200 so that the sela moves with the tubing 200.

The features of the telescopic tubing extension set 100 provide a fluid pathway having a first length in the first configuration, and a second length in the second configuration, where the second length is greater than the first length. Thus, if the tubing 200, or another portion of the telescopic tubing extension set 100 or other device forming the fluid pathway, is pulled to an extend that the force exceeds the threshold force to separate the tubing 200 from the tubing coupler 19, then the tubing 200 can move longitudinally within the sleeve to increase the length of the fluid pathway. By increasing the length of the fluid pathway in the second orientation, the telescopic tubing extension set 100 resists breaking or separation of any portion of the telescopic tubing extension set 100 or IV set that forms the fluid pathway. As a result, the telescopic tubing extension set 100 can maintain the structural integrity and sterility of the fluid pathway.

The telescopic tubing extension set 100 is also configured such that the tubing 200 and sleeve 102 can be moved toward each other to move from the second configuration toward the first configuration. After the telescopic tubing extension set 100 is moved to the second configuration, such as when the tubing 200 has been pulled, the tubing 200 can be moved longitudinally back toward the tubing coupler 19 until the distal end 202 of the tubing is positioned within a fluid channel 22.

The structural integrity and sterility of the fluid pathway is maintained when the telescopic tubing extension set 100 moves between the first and second orientations. By maintaining the structural integrity and sterility of the fluid pathway, the telescopic tubing extension set 100 can reduce labor and cost associated with fluid transfer.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A telescopic tubing extension set comprising: a sleeve having a first end, a second end, and an inner surface forming a tubing lumen extending through the first and second ends of the sleeve, the second end of the sleeve comprising a locking feature configured to couple with a tubing coupler, and the first end of the sleeve comprising a seal; and a tubing having a proximal end, a distal end, an inner surface forming a fluid passage extending through the proximal and distal ends, a collar extending radially outward from an outer surface of the tubing, and a distal end portion formed by the outer surface of the tubing defining a cross-sectional width of the tubing that decreases in a direction from the collar toward the distal end of the tubing, wherein a distal end portion of the tubing is positioned within the tubing lumen of the sleeve such that the seal is engaged against the tubing and the tubing is longitudinally movable relative to the sleeve.

Clause 2. The telescopic tubing extension set of Clause 1, wherein the locking feature comprises a thread extending radially inward from the inner surface of the sleeve and configured to matingly engage a thread of the tubing coupler.

Clause 3. The telescopic tubing extension set of any of the preceding Clauses, wherein an outer surface of the collar defines a first cross-sectional width, and an inner surface of the seal forms an opening having a second cross-sectional width, and wherein the first cross-sectional width is greater than the second cross-sectional width.

Clause 4. The telescopic tubing extension set of Clause 3, wherein the inner surface of the sleeve defines a third cross-sectional width, and wherein the third cross-sectional width is greater than the first cross-sectional width.

Clause 5. The telescopic tubing extension set of any of the preceding Clauses, wherein the seal is engaged against the inner surface of the sleeve and the outer surface of the tubing to resist movement of fluid therebetween.

Clause 6. The telescopic tubing extension set of any of the preceding Clauses, wherein the seal is an o-ring.

Clause 7. The telescopic tubing extension set of any of the preceding Clauses, wherein the distal end portion forms a male luer taper configured to be received within a female luer taper of the tubing coupler to form a luer-slip therebetween.

Clause 8. The telescopic tubing extension set of any of the preceding Clauses, wherein the sleeve comprises a first length from the seal to the second end, and the tubing comprises a second length from the proximal end to the collar, and wherein the first length is less than the second length.

Clause 9. A telescopic tubing extension set system comprising: a tubing coupler having an outer surface and an inner surface, wherein the inner surface forms a fluid channel extending through the tubing coupler; and a telescopic tubing extension set comprising a sleeve, a tubing, and a seal, the sleeve comprising a first end engaged against the seal, a second end having a locking feature configured for coupling with the tubing coupler, and a tubing lumen extending through the first and second ends of the sleeve, and the tubing positioned within the tubing lumen and having a distal end configured for removably coupling with the fluid channel of the tubing coupler, an inner surface forming a fluid passage, and an outer surface engaged against the seal, wherein, in a first orientation of the telescopic tubing extension set, the locking feature is engaged against the tubing coupler to resist separation of the sleeve from the tubing coupler, and the distal end of the tubing is positioned within the fluid channel of the tubing coupler to form a fluid pathway between the fluid channel of the tubing coupler and the fluid passage of the tubing, and in a second orientation of the telescopic tubing extension set, the tubing is longitudinally moved in a direction away from the tubing coupler such that the distal end of the tubing is withdrawn from the tubing coupler and positioned between the first and second ends of the sleeve to form a fluid pathway between the fluid channel of the tubing coupler, the tubing lumen of the sleeve, and the fluid passage of the tubing.

Clause 10. The telescopic tubing extension set of Clause 9, wherein the tubing comprises a collar extending radially outward from the outer surface of the tubing.

Clause 11. The telescopic tubing extension set of Clause 10, wherein the sleeve comprises a first length from the seal to the second end, and the tubing comprises a second length from the collar to a proximal end of the tubing, and wherein the first length is less than the second length.

Clause 12. The telescopic tubing extension set of Clause 10, wherein the seal resists movement of the tubing in the direction away from the tubing coupler when the collar is engaged against the seal.

Clause 13. The telescopic tubing extension set of Clause 10, wherein an outer surface of the collar defines a first cross-sectional width, and an inner surface of the seal forms an opening having a second cross-sectional width, and wherein the first cross-sectional width is greater than the second cross-sectional width.

Clause 14. The telescopic tubing extension set of Clause 13, wherein the inner surface of the sleeve defines a third cross-sectional width, and wherein the third cross-sectional width is greater than the first cross-sectional width.

Clause 15. The telescopic tubing extension set of any of Clauses 9 to 14, wherein a luer-slip is formed between the outer surface of the tubing and the inner surface of the tubing coupler.

Clause 16. The telescopic tubing extension set of any of Clauses 9 to 15, wherein a luer lock is formed between the locking feature of the sleeve and the tubing coupler.

Clause 17. The telescopic tubing extension set of any of Clauses 9 to 16, wherein the locking feature comprises a thread extending radially inward from the inner surface of the sleeve and configured to engage a mating thread of the tubing coupler.

Clause 18. A method for providing a telescopic tubing extension set, the method comprising: providing a sleeve with an inner surface forming a tubing lumen between a first end and a second end of the sleeve; positioning a tubing within the tubing lumen of the sleeve such that tubing is longitudinally movable within the tubing lumen in a first direction, such that a distal end of the tubing moves toward the second end of the sleeve, and a second direction, such that a distal end of the tubing moves away the second end of the sleeve; and positioning a seal between the sleeve and the tubing to form resist movement of a fluid between an outer surface of the tubing and the inner surface of the sleeve.

Clause 19. The method of Clause 18, wherein positioning the seal between the sleeve and the tubing comprises inserting the seal into the tubing lumen of the sleeve such that an opening through the seal is coaxial with the tubing lumen, and inserting the tubing through the opening of the seal such the seal is engaged against the inner surface of the sleeve and the outer surface of the tubing.

Clause 20. The method of any of Clauses 18 and 19, wherein positioning the tubing within the tubing lumen of the sleeve comprises orienting the tubing so that a collar, extending radially outward from the outer surface of the tubing, is positioned between the seal and the second end of the sleeve.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A telescopic tubing extension set comprising:
a sleeve having a first end, a second end, and an inner surface forming a tubing lumen extending through the first and second ends of the sleeve, the second end of the sleeve comprising a locking feature configured to couple with a tubing coupler, and the first end of the sleeve comprising a seal; and
a tubing having a proximal end, a distal end, an inner surface forming a fluid passage extending through the proximal and distal ends, a collar extending radially outward from an outer surface of the tubing, and a distal end portion formed by the outer surface of the tubing defining a cross-sectional width of the tubing that decreases in a direction from the collar toward the distal end of the tubing,
wherein a distal end portion of the tubing is positioned within the tubing lumen of the sleeve such that the seal is engaged against the tubing and the tubing is longitudinally movable relative to the sleeve.

2. The telescopic tubing extension set of claim 1, wherein the locking feature comprises a thread extending radially inward from the inner surface of the sleeve and configured to matingly engage a thread of the tubing coupler.

3. The telescopic tubing extension set of claim 1, wherein an outer surface of the collar defines a first cross-sectional width, and an inner surface of the seal forms an opening having a second cross-sectional width, and wherein the first cross-sectional width is greater than the second cross-sectional width.

4. The telescopic tubing extension set of claim 3, wherein the inner surface of the sleeve defines a third cross-sectional width, and wherein the third cross-sectional width is greater than the first cross-sectional width.

5. The telescopic tubing extension set of claim 1, wherein the seal is engaged against the inner surface of the sleeve and the outer surface of the tubing to resist movement of fluid therebetween.

6. The telescopic tubing extension set of claim 1, wherein the seal is an o-ring.

7. The telescopic tubing extension set of claim 1, wherein the distal end portion forms a male luer taper configured to be received within a female luer taper of the tubing coupler to form a luer-slip therebetween.

8. The telescopic tubing extension set of claim 1, wherein the sleeve comprises a first length from the seal to the second end, and the tubing comprises a second length from the proximal end to the collar, and wherein the first length is less than the second length.

9. A telescopic tubing extension set system comprising:
a tubing coupler having an outer surface and an inner surface, wherein the inner surface forms a fluid channel extending through the tubing coupler; and
a telescopic tubing extension set comprising a sleeve, a tubing, and a seal, the sleeve comprising a first end engaged against the seal, a second end having a locking feature configured for coupling with the tubing coupler, and a tubing lumen extending through the first and second ends of the sleeve, and the tubing positioned within the tubing lumen and having a distal end configured for removably coupling with the fluid channel of the tubing coupler, an inner surface forming a fluid passage, and an outer surface engaged against the seal,
wherein, in a first orientation of the telescopic tubing extension set, the locking feature is engaged against the tubing coupler to resist separation of the sleeve from the tubing coupler, and the distal end of the tubing is positioned within the fluid channel of the tubing coupler to form a fluid pathway between the fluid channel of the tubing coupler and the fluid passage of the tubing, and in a second orientation of the telescopic tubing extension set, the tubing is longitudinally moved in a direction away from the tubing coupler such that the distal end of the tubing is withdrawn from the tubing coupler and positioned between the first and second ends of the sleeve to form a fluid pathway between the fluid channel of the tubing coupler, the tubing lumen of the sleeve, and the fluid passage of the tubing.

10. The telescopic tubing extension set of claim 9, wherein the tubing comprises a collar extending radially outward from the outer surface of the tubing.

11. The telescopic tubing extension set of claim 10, wherein the sleeve comprises a first length from the seal to the second end, and the tubing comprises a second length from the collar to a proximal end of the tubing, and wherein the first length is less than the second length.

12. The telescopic tubing extension set of claim 10, wherein the seal resists movement of the tubing in the direction away from the tubing coupler when the collar is engaged against the seal.

13. The telescopic tubing extension set of claim 10, wherein an outer surface of the collar defines a first cross-sectional width, and an inner surface of the seal forms an opening having a second cross-sectional width, and wherein the first cross-sectional width is greater than the second cross-sectional width.

14. The telescopic tubing extension set of claim 13, wherein the inner surface of the sleeve defines a third cross-sectional width, and wherein the third cross-sectional width is greater than the first cross-sectional width.

15. The telescopic tubing extension set of claim 9, wherein a luer-slip is formed between the outer surface of the tubing and the inner surface of the tubing coupler.

16. The telescopic tubing extension set of claim 9, wherein a luer lock is formed between the locking feature of the sleeve and the tubing coupler.

17. The telescopic tubing extension set of claim 9, wherein the locking feature comprises a thread extending radially inward from the inner surface of the sleeve and configured to engage a mating thread of the tubing coupler.

18. A method for providing a telescopic tubing extension set, the method comprising:
providing a sleeve with an inner surface forming a tubing lumen between a first end and a second end of the sleeve;
positioning a tubing within the tubing lumen of the sleeve such that tubing is longitudinally movable within the tubing lumen in a first direction, such that a distal end of the tubing moves toward the second end of the sleeve, and a second direction, such that a distal end of the tubing moves away the second end of the sleeve; and
positioning a seal between the sleeve and the tubing to form resist movement of a fluid between an outer surface of the tubing and the inner surface of the sleeve.

19. The method of claim 18, wherein positioning the seal between the sleeve and the tubing comprises inserting the seal into the tubing lumen of the sleeve such that an opening through the seal is coaxial with the tubing lumen, and inserting the tubing through the opening of the seal such the seal is engaged against the inner surface of the sleeve and the outer surface of the tubing.

20. The method of claim 18, wherein positioning the tubing within the tubing lumen of the sleeve comprises orienting the tubing so that a collar, extending radially outward from the outer surface of the tubing, is positioned between the seal and the second end of the sleeve.
